(19) United States
(12) Patent Application Publication
(10) Pub. No.: US 2005/0165047 A1
(43) Pub. Date: Jul. 28, 2005

(54) Crystalline fumarate salts of 1-azabicyclo[2.2.2]oct substituted furo[2,3-c]pyridinyl carboxamide and compositions and preparations thereof

(75) Inventors: Jon Gordon Selbo, Wentzville, MO (US); Donn Gregory Wishka, Mystic, CT (US); Bradley D. Hewitt, Kalamazoo, MI (US); David Warner Rappath, Kalamazoo, MI (US); Ahmad Sheikh, Deerfield, IL (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/962,071

(22) Filed: Oct. 8, 2004

Related U.S. Application Data
(63) Continuation of application No. 10/729,286, filed on Dec. 5, 2003, now abandoned.
(60) Provisional application No. 60/431,619, filed on Dec. 6, 2002.

(51) Int. Cl. C07D 491/02; A61K 31/4745

(52) U.S. Cl. 514/302; 546/114

(57) ABSTRACT

The invention provides fumarate salts of N-[1-azabicyclo[2.2.2]oct-3-yl]furo[2,3-c]pyridine-5-carboxamide, compositions, racemic mixtures, or pure enantiomers thereof, and preparation thereof. The fumarate salts are useful to treat diseases or conditions in which α7 nAChR is known to be involved.

### CROSS REFERENCE TO RELATED APPLICATIONS

This application claims the benefit of U.S. provisional application Ser. No. 60/431,619 filed on 6 Dec. 2002, under 35 USC 119(e)(i), which is incorporated herein by reference in its entirety.

### FIELD OF INVENTION

The present invention relates to crystals, and compositions thereof, wherein the crystals include the fumarate salts of N-[1-azabicyclo[2.2.2]oct-3-yl]furo[2,3-c]pyridine-5-carboxamide. The present invention also relates to methods of preparing such crystals.

### BACKGROUND OF THE INVENTION

Nicotinic acetylcholine receptors (nAChRs) play a large role in central nervous system (CNS) activity. Particularly, they are known to be involved in cognition, learning, mood, emotion, and neuroprotection. There are several types of nicotinic acetylcholine receptors, and each one appears to have a different role in regulating CNS function. Nicotine affects all such receptors, and has a variety of activities. Unfortunately, not all of the activities are desirable. In fact, one of the least desirable properties of nicotine is its addictive nature and the low ratio between efficacy and safety. The present invention relates to molecules that have a greater effect upon the α7 nAChRs as compared to other closely related members of this large ligand-gated receptor family. Thus, the invention provides the stable fumarate salts of N-[(3R)-1-azabicyclo[2.2.2]oct-3-yl]furo[2,3-c]pyridine-5-carboxamide that are active drug molecules with fewer side effects.

Cell surface receptors are, in general, excellent and validated drug targets. nAChRs comprise a large family of ligand-gated ion channels that control neuronal activity and brain function. These receptors have a pentameric structure. In mammals, this gene family is composed of nine alpha and four beta subunits that co-assemble to form multiple subtypes of receptors that have a distinctive pharmacology. Acetylcholine is the endogenous regulator of all of the subtypes, while nicotine non-selectively activates all nAChRs.

The α7 nAChR is one receptor system that has proved to be a difficult target for testing. Native α7 nAChR is not routinely able to be stably expressed in most mammalian cell lines (Cooper and Millar, J. Neurochem., 1997, 68 (5):2140-51). Another feature that makes functional assays of α7 nAChR challenging is that the receptor is rapidly (100 milliseconds) inactivated. This rapid inactivation greatly limits the functional assays that can be used to measure channel activity.

Recently, Eisele et al. has indicated that a chimeric receptor formed between the N-terminal ligand binding domain of the α7 nAChR (Eisele et al., Nature, 366(6454), p 479-83, 1993), and the pore forming C-terminal domain of the 5-HT₃ receptor expressed well in Xenopus oocytes while retaining nicotinic agonist sensitivity. Eisele et al. used the N-terminus of the avian (chick) form of the α7 nAChR receptor and the C-terminus of the mouse form of the 5-HT₃ gene. However, under physiological conditions the α7 nAChR is a calcium channel while the 5-HT₃R is a sodium and potassium channel. Indeed, Eisele et al. teaches that the chicken α7 nAChR/mouse 5-HT₃R behaves quite differently than the native α7 nAChR with the pore element not conducting calcium but actually being blocked by calcium ions. WO 00/73431 A2 reports on assay conditions under which the 5-HT₃R can be made to conduct calcium. This assay may be used to screen for agonist activity at this receptor.

### SUMMARY OF THE INVENTION

The present invention discloses fumarate salts of the Formula I: 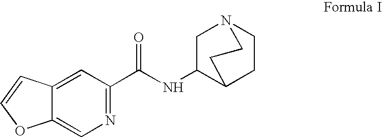
or pharmaceutical composition, racemic mixture, or pure enantiomer thereof, provided that the salt is the fumarate salt thereof. The compound of Formula I is also known as N-[1-azabicyclo[2.2.2]oct-3-yl]furo[2,3-c]pyridine-5-carboxamide. Formula I can be a pure enantiomer, for example, but not limitation, N-[(3R)-1-azabicyclo[2.2.2]oct-3yl]furo[2,3-c]pyridine-5-carboxamide of varying degrees of enantiomeric purity. The present invention includes fumarate salts of varying ratios of fumarate salt equivalents. For example, but not by limitation, one aspect of the present invention includes one equivalent of fumarate salt, the mono-fumarate salt. Another aspect of the present invention includes one-half equivalent of fumarate salt, the hemi-fumarate salt. One equivalent of fumarate salt is preferred.

The present invention includes the fumurate salts of Formula I. Surprisingly, the fumarate salts of Formula I are crystalline, are relatively non-hygroscopic, and generally have better physical properties than other salts, including a melting point above that of the free base. Another aspect of the present invention includes the anhydrous crystal form of the fumarate salts. The present invention also includes the method of preparing the fumarate salts of Formula I.

In another aspect, the present invention provides methods of preparing a crystal including a mono fumarate salt of N-[(3R)-1-azabicyclo[2.2.2]oct-3-yl]furo[2,3-c]pyridine-5-carboxamide (e.g., Crystal form Ia). In one embodiment, the method includes dissolving the free base in an alcohol, by heating, for example, but not by way of limitation, over a steam bath, adding at least 1 eq of fumaric acid, and allowing the reaction to cool from about room temperature to about −20° C. whereby the salt precipitates out of solution. Another aspect includes dissolving the free base in an alcohol, preferably methanol or ethanol, to give a concentration from about 0.04M to about 1M.

The method of making the mono-fumarate salt also includes dissolving the free base in isopropanol to give a concentration of about 0.1M to about 1M, adding a solution of at least 1 eq of fumaric acid dissolved in methanol to give a concentration of about 2M to about 5M, and adding acetone to the fumaric acid solution to give a final concentration of about 0.1M to about 0.5M, stirring the reaction for about 1 to 3 hours, adding acetone to give a final concentration of about 0.05M to about 0.2M, stirring, optionally, for about 8-24 h, collecting and washing the solid with fresh acetone, and drying the salt.

The method of making the mono-fumarate salt also includes dissolving the free base in isopropanol to give a concentration of about 0.25M to about 0.75M (or any range therein, e.g, 0.4 to 0.6), adding a solution of at least 1 eq of fumaric acid dissolved in methanol to give a concentration of about 3M to about 4M, and adding acetone to the fumaric acid solution to give a final concentration of about 0.25M to about 0.35M, stirring the reaction for about 2 hours, adding acetone to give a final concentration of about 0.1M, stirring, optionally, about 12-20 hours (or any range therein, e.g., 14 to 16 hours), collecting and washing the solid with fresh acetone, and drying the salt.

The method also includes dissolving the free base in n-butanol to give a solution of about 0.6M to about 0.8M. Adding the solution containing the free base to about 0.35M to about 0.45M solution of at least 1 eq fumaric acid in 30% water/acetone. The solution is then concentrated to about 0.55M to about 0.75M by vacuum distillation. n-Butanol is added to give a concentration of the free base from about 0.4M to about 0.6M. The slurry removed, and the resulting fumarate salt is rinsed with n-butanol and dried, optionally, for about 2 to 5 days in an 80° C. vacuum oven.

The present invention also includes the method of preparing the mono-fumarate salt, comprising dissolving the free base in an alcohol (including methanol or ethanol) to give a concentration from about 0.04M to about 1M by heating, for example, but not by limitation, over a steam bath; adding at least 1 eq of fumaric acid; allowing the reaction to cool from about room temperature to about −20° C. whereby the salt precipitates out of solution; and collecting and drying the salt.

The present invention also includes the method of preparing the fumarate salt, comprising dissolving the free base in an alcohol (including isopropanol) to give a concentration of about 0.1M to about 1M (and ranges therein, including about 0.4 M to about 0.8M and ranges therein, e.g., about 0.5M to about 0.6M); adding a solution of at least 1 eq of fumaric acid dissolved in an alcohol (including methanol or ethanol) to give a concentration of about 2M to about 5M (including about 3M to about 4M and ranges therein) and adding acetone to the fumaric acid solution to give a final concentration of about 0.1M to about 0.5M (including about 0.25M to about 0.4M and ranges therein); and stirring the reaction for about 1 to about 3 hours, adding acetone to give a final concentration of about 0.05 M to about 0.2M (including about 0.075M to about 0.15M and ranges therein), stirring, optionally, about 8-24 h, collecting and optionally washing the solid with acetone (fresh), and drying the salt.

The present invention also includes the method of preparing the mono-fumarate salt, comprising dissolving the free base in an alcohol (including n-butanol) to give a solution of about 0.6M to about 0.8M (including about 0.7M); adding the solution containing the free base to about 0.35M to about 0.45M solution (including a 0.4M solution) of at least 1 eq fumaric acid in 30% water/acetone; concentrating the reaction to about 0.55M to about 0.75M (including using vacuum distillation); adding more alcohol to give a concentration of the free base from about 0.4M to about 0.6M; removing a resulting solid, rinsing with alcohol, and drying for, optionally, about 2 to 5 days (including 3 days) optionally drying with heat. The heat can be at about 80° C.

Another aspect of the present invention provides methods of preparing a crystal including a hemi-fumarate salt of N-[(3R)-1-azabicyclo[2.2.2]oct-3-yl]furo[2,3-c]pyridine-5-carboxamide (e.g., Crystal Form Ib). The hemi-fumarate salt has a stoichiometric value of 0.5 equivalents for each equivalent of free base. In one embodiment, the method includes dissolving the free base in isopropanol (IPA) at approximately 9 wt % by heating, for example, but not by limitation, to approximately 70° C. A separate solution of fumaric acid in IPA (2.8 wt %) is prepared with approximately (e.g., +/−10%) 0.5 molar equivalents of fumaric acid by heating to approximately 70° C. The IPA/free-base solution is then added to the IPA/fumaric acid solution, or the IPA/fumaric acid solution is added to the IPA/free-base solution, while maintaining the temperature. Precipitation commences immediately after the addition is complete. The system is held at approximately 70° C., before allowing the system to cool to room temperature, at which temperature the slurry is filtered, the cake washed with IPA and then oven dried at about 45° C. under 28 inches of Hg.

The present invention also includes a method for treating, or using the fumarate salts of formula I to prepare a medicament to treat, a disease or condition in a mammal in need thereof, wherein the mammal would receive symptomatic relief from the administration of a fumarate salt of formula I.

The present invention also includes a method for treating, or using the fumarate salts of formula I to prepare a medicament to treat, a disease or condition in a mammal in need thereof comprising administering to the mammal a therapeutically effective amount of a fumarate salt of Formula I, wherein the disease or condition is any one or more or combination of the following: cognitive and attention deficit symptoms of Alzheimer's Disease, neurodegeneration associated with diseases such as Alzheimer's disease, pre-senile dementia (mild cognitive impairment), senile dementia, schizophrenia, psychosis, attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems in general and associated with brain tumors, AIDS dementia complex, dementia associated with Down's syndrome, dementia associated with Lewy Bodies, Huntington's disease, Parkinson's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, or symptoms associated with pain.

In another aspect, the invention includes treating a mammal suffering from schizophrenia or psychosis by administering a fumarate salt of Formula I in conjunction with antipsychotic drugs (also called anti-psychotic agents). The compounds of the present invention and the antipsychotic drugs can be administered simultaneously or at separate intervals. When administered simultaneously the compounds of the present invention and the antipsychotic drugs can be incorporated into a single pharmaceutical composition. Alternatively, two separate compositions, i.e., one containing compounds of the present invention and the other containing antipsychotic drugs, can be administered simultaneously.

Numerous factors affect crystallization conditions, and they are well known to one of ordinary skill in the art. Such factors include, for example, but not by way of limitation: the concentration of the salt in the crystallization solution; the difference, if any, between the initial and final temperatures of the crystallization solution; the rate of cooling, if any; the solvent vaporization rate, if any; seeding; supersaturation ratio; and presence of a precipitant. With guidance from the disclosure provided herein, one of ordinary skill in the art, without undue experimentation, may select and/or adjust one or more appropriate factors to arrive at crystallization conditions. Useful solvents for the crystallization solution include, for example, but are not limited to, any one of the following: methanol, ethanol, isopropanol, n-butanol, ethyl acetate, ether, dimethyl ketone, water.

Further aspects and embodiments of the invention may become apparent to those skilled in the art from a review of the following detailed description, taken in conjunction with the examples and the claims. While the invention is susceptible of embodiments in various forms, described hereafter are specific embodiments of the invention with the understanding that the present disclosure is intended as illustrative, and is not intended to limit the invention to the specific embodiments described herein.

### DETAILED DESCRIPTION OF THE INVENTION

Surprisingly, we have found that the fumarate salts of the Formula I: 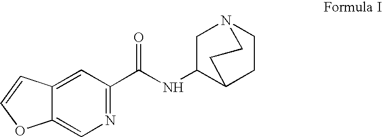
or pharmaceutical composition, racemic mixture, or pure enantiomer thereof, provided that the salt is the fumarate salt thereof, are crystalline, are relatively non-hygroscopic, and generally have better physical properties than other salts.

The present invention also includes the processes to make the fumarate salts and the fumarate salts of Formula I, pharmaceutical compositions containing them, and methods to treat the identified diseases using the fumarate salts of Formula I.

The compounds of Formula I have an optically active center on the quinuclidine ring. Although it is desirable that the stereochemical purity be as high as possible, absolute purity is not required. This invention involves racemic mixtures and compositions of varying degrees of streochemical purities. It is preferred to carry out stereoselective syntheses and/or to subject the reaction product to appropriate purification steps so as to produce substantially enantiomerically pure materials. Suitable stereoselective synthetic procedures for producing enantiomerically pure materials are well known in the art, as are procedures for purifying racemic mixtures into enantiomerically pure fractions.

Abbreviations which are well known to one of ordinary skill in the art may be used (e.g., “Me” for methyl, “Et” for ethyl, “h” for hour or hours, min for minute or minutes, and “rt” or “RT” for room temperature).

All temperatures are in degrees Centigrade.

Room temperature is within the range of 15-25 degrees Celsius.

Pre-senile dementia is also known as mild cognitive impairment.

ACHR refers to acetylcholine receptor.

nAChR refers to nicotinic acetylcholine receptor.

5HT₃R refers to the serotonin-type 3 receptor.

α-btx refers to α-bungarotoxin.

FLIPR refers to a device marketed by Molecular Devices, Inc. designed to precisely measure cellular fluorescence in a high throughput whole-cell assay. (Schroeder et. al., J. Biomolecular Screening, 1 (2), p 75-80, 1996).

TLC refers to thin-layer chromatography.

HPLC refers to high pressure liquid chromatography.

MeOH refers to methanol.

EtOH refers to ethanol.

IPA refers to isopropyl alcohol.

THF refers to tetrahydrofuran.

DMSO refers to dimethylsulfoxide.

DMF refers to dimethylformamide.

EtOAc refers to ethyl acetate.

TMS refers to tetramethylsilane.

TEA refers to triethylamine.

DIEA refers to diisopropylethylamine.

MLA refers to methyllycaconitine.

Ether refers to diethyl ether.

HATU refers to O-(7-azabenzotriazol-1-yl)-N,N,N′,N′-tetramethyluronium hexafluorophosphate.

DBU refers to 1,8-diazabicyclo[5.4.0]undec-7-ene.

50% saturated 1:1 NaCl/NaHCO₃ means a solution made by making a solution of 1:1 saturated NaCl/NaHCO₃ and adding an equal volume of water.

Na₂SO₄ refers to sodium sulfate.

K₂CO₃ refers to potassium carbonate.

MgSO₄ refers to magnesium sulfate.

When Na₂SO₄, K₂CO₃, or MgSO₄ is used as a drying agent, it is anhydrous.

NaHCO₃ refers to sodium bicarbonate.

KHCO₃ refers to potassium bicarbonate.

(2E)-but-2-enedioic acid is used interchangeably with fumarate salt. Both mean the same salt.

Mammal denotes a human being, and other mammals and animals, such as food animals (e.g., cows, pigs, sheep, goats, deer, poultry, etc.), companion animals (e.g., dogs, cats, horses, birds, and fish), or other mammals.

Brine refers to an aqueous saturated sodium chloride solution.

Equ means molar equivalents.

IR refers to infrared spectroscopy.

PSI means pound per square inch.

NMR refers to nuclear (proton) magnetic resonance spectroscopy, chemical shifts are reported in ppm (δ) downfield from TMS.

MS refers to mass spectrometry expressed as m/e or mass/charge unit. HRMS refers to high resolution mass spectrometry expressed as m/e or mass/charge unit. [M+H]⁺ refers to an ion composed of the parent plus a proton. [M−H]⁻ refers to an ion composed of the parent minus a proton. [M+Na]⁺ refers to an ion composed of the parent plus a sodium ion. [M+K]⁺ refers to an ion composed of the parent plus a potassium ion. EI refers to electron impact. ESI refers to electrospray ionization. CI refers to chemical ionization. FAB refers to fast atom bombardment.

As used herein, “supersaturation ratio” refers to the ratio of the concentration of the material in solution to the concentration of the material in a saturated solution at the crystallization temperature.

As used herein, “seeding” refers to the technique of adding a “seed” crystal to the crystallization solution to promote the formation of crystals. Preferably, the composition of the seed crystal is the same as the composition of the crystals being formed.

As used herein, “precipitant” means a substance that tends to induce crystallization when added to a crystallization solution. Useful precipitants include, for example, non-solvents for the salt and solutions including excess counterions. As used herein, a non-solvent is a solvent in which the salt preferably has a solubility of at most about 1% by weight, more preferably at most about 0.1% by weight, and most preferably at most about 0.01% by weight.

As used herein, “anhydrous crystal” means a crystal in which water is not specifically bound. Anhydrous crystals preferably do not include substantial amounts of water. The water content can be determined by methods known in the art including, for example, Karl Fischer titrations. Preferably an anhydrous crystal includes at most about 2% by weight water, more preferably at most about 0.5% by weight water, and most preferably less than about 0.3% by weight water.

As used herein, “crystalline” means a material that has an ordered, long range molecular structure. The degree of crystallinity of a crystal form can be determined by many techniques including, for example, powder X-ray diffraction, moisture sorption, differential scanning calorimetry, solution calorimetry, and dissolution properties.

As used herein, “more crystalline” means that a material has a higher degree of crystallinity than the material to which it is being compared. Materials with higher degrees of crystallinity generally have highly ordered, long range molecular structure with fewer defects in the crystal structure than materials with lower degrees of crystallinity. The higher degree of crystallinity can be assessed relative to the other form by techniques including, for example, sharper reflections in the powder X-ray diffraction pattern, lower moisture sorption for similar sized particles at a specified relative humidity, lower heat of solution, higher heat of fusion, slower dissolution rate, and combinations thereof.

As used herein, “less crystalline” means that a material has a lower degree of crystallinity than the material to which it is being compared. Materials with lower degrees of crystallinity generally have less long range order and more defects in the crystal structure than materials with higher degrees of crystallinity. The lower degree of crystallinity can be assessed relative to the other form by techniques including, for example, broader and/or fewer reflections in the powder X-ray diffraction pattern, higher moisture sorption for similar sized particles at a specified relative humidity, higher heat of solution, lower heat of fusion, faster dissolution rate, and combinations thereof.

As referred to in the present application, “stable” in bulk drug stability tests means that at least about 97% by weight, preferably at least about 98% by weight, and more preferably at least about 99% by weight of the bulk drug remains unchanged after storage under the indicated conditions for the indicated time.

Powder X-Ray Diffraction (PXRD)

Crystalline organic compounds consist of a large number of atoms that are arranged in a periodic array in three-dimensional space. The structural periodicity normally manifests distinct physical properties, such as sharp, explicit spectral features by most spectroscopic probes (e.g., X-ray diffraction, infrared and solid state NMR). X-ray diffraction (XRD) is acknowledged to be one of the most sensitive methods to determine the crystallinity of solids. Crystals yield explicit diffraction maxima that arise at specific angles consistent with the lattice interplanar spacings, as predicted by Bragg's law. On the contrary, amorphous materials do not possess long-range order. They often retain additional volume between molecules, as in the liquid state. Amorphous solids normally unveil a featureless XRD pattern with broad, diffuse halos because of the absence of the long range order of repeating crystal lattice.

PXRD has reportedly been used to characterize different crystal forms of organic compounds (e.g., compounds useful in pharmaceutical compositions). See, for example, U.S. Pat. No. 5,504,216 (Holohan et al), U.S. Pat. No. 5,721,359 (Dunn et al.), U.S. Pat. No. 5,910,588 (Wangnick et al.), U.S. Pat. No. 6,066,647 (Douglas et al.), U.S. Pat. No. 6,225,474 (Matsumoto et al.), U.S. Pat. No. 6,239,141 (Allen et al.), U.S. Pat. No. 6,251,355 (Murata et al.), U.S. Pat. No. 6,288,057 (Harkness), U.S. Pat. No. 6,316,672 (Stowell et al.), and U.S. Pat. No. 6,329,364 (Groleau).

Crystalline materials are preferred in many pharmaceutical applications. Crystalline forms are generally thermodynamically more stable than amorphous forms of the same substance. This thermodynamic stability is preferably reflected in the lower solubility and improved physical stability of the crystalline form. The regular packing of the molecules in the crystalline solid preferably denies the incorporation of chemical impurities. Hence crystalline materials generally possess higher chemical purity than their amorphous counterparts. The packing in the crystalline solid generally constrains the molecules to well defined lattice positions and reduces the molecular mobility that is the prerequisite for chemical reactions. Hence, crystalline solids, with very few notable exceptions, are chemically more stable than amorphous solids of the same molecular composition. Preferably, the crystalline forms of fumarate salts of N-[1-azabicyclo[2.2.2]oct-3-yl]furo[2,3-c]pyridine-5-carboxamide disclosed in the present application possess one or more of the advantageous chemical and/or physical properties disclosed herein.

The crystalline forms of fumarate salts of N-[1-azabicyclo[2.2.2]oct-3-yl]furo[2,3-c]pyridine-5-carboxamide disclosed in the present application preferably have distinct powder X-ray diffraction profiles. Characteristic diffraction peaks as used herein are peaks selected from the most intense peaks of the observed diffraction pattern. Preferably, the characteristic peaks are selected from about 20 of the most intense peaks, more preferably from about 10 of the most intense peaks, and most preferably from about 4 to 5 of the most intense peaks in the diffraction pattern.

PXRD was performed using a Scintag X1 or X2 Advanced Diffraction System operating under Scintag DMS/NT™ and Microsoft Windows NT™ 4.0 software. The system used a copper X-ray source maintained at 45 kV and 40 mA to provide Cu KL3 (Kα₁) emission of 1.5406 Å and a solid-state peltier cooled detector. Beam aperture was controlled using tube divergence and anti-scatter slits of 2 and 4 mm and detector anti-scatter and receiving slits of 0.5 and 0.3 mm width. Data were collected using a step scan of 0.02° per point with a one-half second per point counting time over a range of 2 to 35° two-theta. Scintag Round, Top Loading stainless steel Sample Cups (Part Number 1ZEO-20-0120-01) were utilized for all analyses. Aluminum spacers with a 12 mm cavity were utilized to accommodate small sample sizes. Samples were run as received or after hand grinding.

Tables 1 and 2 show the powder X-ray diffraction patterns for Crystal Forms Ia and Ib of the mono- and hemi-fumarate salts, respectively, of N-[(3R)-1-azabicyclo[2.2.2]oct-3-yl]furo[2,3-c]pyridine-5-carboxamide. Table 1 contains the listing of the most intense peaks from the PXRD pattern between 2 and 35 degrees two theta for the mono-fumarate salt. Table 2 contains the listing of the most intense peaks from the PXRD pattern between 2 and 35 degrees two theta for the hemi-fumarate salt. The free base, Crystal Form Ia, and Crystal Form Ib are all easily distinguished by their unique PXRD patterns (not shown).

Preferably an anhydrous crystal including a mono-fumarate salt of N-[(3R)-1-azabicyclo[2.2.2]oct-3-yl]furo[2,3-c]pyridine-5-carboxamide (e.g., Crystal Form Ia) has characteristic diffraction peaks at about 18.90 and 24.97 degrees two-theta, more preferably at about 18.21, 18.90, 21.74 and 24.97 degrees two-theta, and most preferably has the characteristic diffraction peaks as listed in Table 1 for Crystal Form Ia:

TABLE 1
Powder X-Ray Diffraction Peak Table for Crystal Form Ia

| Position | |
|---|---|
| (Degrees Two | Relative |
| Theta) | Intensity |
| 13.20 | 22 |
| 15.47 | 14 |
| 18.21 | 39 |
| 18.90 | 76 |
| 19.87 | 20 |
| 20.81 | 21 |
| 21.15 | 16 |
| 21.74 | 30 |
| 21.96 | 23 |
| 24.32 | 12 |
| 24.97 | 100 |
| 26.52 | 27 |
| 28.36 | 15 |
| 28.82 | 20 |
| 29.49 | 14 |

Preferably, Crystal Form Ib of a hemi-fumarate salt of N-[(3R)-1-azabicyclo[2.2.2]oct-3-yl]furo[2,3-c]pyridine-5-carboxamide has characteristic diffraction peaks at about 19.84 and 24.83 degrees two-theta, more preferably at about 17.59, 18.43, 19.84, 22.74, and 24.83 degrees two-theta, and most preferably has the characteristic diffraction peaks as listed in Table 2 for Crystal Form Ib:

TABLE 2
Powder X-Ray Diffraction Peak Table for Crystal Form Ib

| Position | |
|---|---|
| (Degrees Two | Relative |
| Theta) | Intensity |
| 5.00 | 12 |
| 12.59 | 23 |
| 14.58 | 17 |
| 16.23 | 24 |
| 17.59 | 60 |
| 18.43 | 60 |
| 18.96 | 23 |
| 19.50 | 18 |
| 19.84 | 67 |
| 20.31 | 18 |
| 20.85 | 25 |
| 22.11 | 19 |
| 22.74 | 46 |
| 24.83 | 100 |
| 25.34 | 36 |
| 27.92 | 22 |
| 29.13 | 19 |
| 29.87 | 17 |
| 30.15 | 18 |

Moisture Sorption Data

The sorption of water by the solid lattice at a given relative humidity (RH) was measured by DMSG on a controlled atmosphere microbalance. Scans were carried out at 25° C. from 36% to 0% RH then ramped to 90% RH, and back down to 0% RH with a step size of 3% RH. A sample size of 8 to 14 mg was used. At each step, the balance was considered equilibrated when the mass change was less than 0.01 mg for five consecutive scans. There were 120 seconds between each scan.

Table 3 provides the moisture sorption data at 25° C. for Crystal Forms Ia and Ib of N-[1-azabicyclo[2.2.2]oct-3-yl]furo[2,3-c]pyridine-5-carboxamide. The more crystalline polymorph, Crystal Form Ia, is less hygroscopic above 74% relative humidity (RH) than Crystal Form Ib.

TABLE 3
Moisture Sorption of Crystal Forms Ia and Ib
36-0%, 0-90%, and 90-0% Relative Humidity (RH)

| | |
|---|---|
| Crystal Form Ia | Crystal Form Ib |

| RH (%) | % dMass | RH (%) | % dMass |
|---|---|---|---|
| 31.62 | 0.08984726 | 36.54 | 0.461252998 |
| 29.02 | 0.08984726 | 33.94 | 0.43149474 |
| 25.92 | 0.085399376 | 31.08 | 0.400861239 |
| 23.18 | 0.077393184 | 28.16 | 0.377229681 |
| 19.94 | 0.070276569 | 25.1 | 0.34659618 |
| 17.1 | 0.062270378 | 21.88 | 0.319463651 |
| 13.96 | 0.055153763 | 18.8 | 0.293206365 |
| 10.8 | 0.048926726 | 15.78 | 0.267824321 |
| 7.98 | 0.042699688 | 12.78 | 0.245068006 |
| 4.74 | 0.033803919 | 9.74 | 0.219685963 |
| 1.6 | 0.022239421 | 6.36 | 0.189927705 |
| 0.36 | 0.011564499 | 3.16 | 0.162795176 |
| 0.1 | 0.012454076 | 0.24 | 0.122534003 |
| 0 | 0.008895768 | 0 | 0.116407303 |
| 0 | 0.006227038 | 2.62 | 0.147916047 |
| 1.3 | 0.015122806 | 5.84 | 0.176799062 |
| 4.3 | 0.025797728 | 8.98 | 0.204806834 |
| 7.1 | 0.037362227 | 12.2 | 0.229313635 |
| 10.1 | 0.044478841 | 15.34 | 0.254695678 |
| 13.14 | 0.052485033 | 18.46 | 0.280952964 |
| 16.18 | 0.057822494 | 21.42 | 0.303709279 |
| 19.28 | 0.062270378 | 24.66 | 0.333467537 |
| 22.56 | 0.0729453 | 27.74 | 0.360600067 |
| 25.72 | 0.078282761 | 30.82 | 0.39210881 |
| 28.68 | 0.083620222 | 33.92 | 0.424492797 |
| 31.96 | 0.093405567 | 36.96 | 0.458627269 |
| 35.18 | 0.100522182 | 39.9 | 0.491011256 |
| 38.16 | 0.107638796 | 43.18 | 0.5347734 |
| 41.24 | 0.113865834 | 46.14 | 0.576785058 |
| 44.36 | 0.116534565 | 49.16 | 0.630174874 |
| 47.56 | 0.123651179 | 52.12 | 0.687065661 |
| 50.7 | 0.133436524 | 55.1 | 0.750083148 |
| 53.86 | 0.142332293 | 58.08 | 0.821853064 |
| 56.98 | 0.153007214 | 61.08 | 0.906751624 |
| 60.24 | 0.164571713 | 64.04 | 1.002153097 |
| 63.9 | 0.184142403 | 67.02 | 1.123811858 |
| 66.66 | 0.202823517 | 70 | 1.281355576 |
| 69.48 | 0.225062938 | 72.9 | 1.505417753 |
| 72.76 | 0.256198127 | 75.9 | 1.924659093 |
| 75.68 | 0.293560353 | 79.2 | 26.9268472 |
| 78.42 | 0.337149618 | 82 | 27.40297933 |
| 81.9 | 0.416321956 | 85.04 | 28.18457122 |
| 85.3 | 0.621814203 | 87.82 | 30.2886551 |
| 88.38 | 0.964301282 | 90.7 | 38.77851104 |
| 88.42 | 0.972307473 | 90.7 | 38.79951687 |
| 85.94 | 0.692980349 | 87.9 | 32.20981322 |
| 83.48 | 0.556875095 | 85.12 | 30.2028813 |
| 80.84 | 0.470586142 | 82.5 | 27.99551876 |
| 77.96 | 0.407426187 | 79.88 | 27.35834194 |
| 75.26 | 0.362947346 | 77.1 | 26.91634429 |
| 72.34 | 0.319358081 | 74.3 | 26.57762529 |
| 69.18 | 0.233958706 | 71.48 | 26.27216553 |
| 66.22 | 0.204602671 | 68.62 | 26.01134315 |
| 63.18 | 0.18236325 | 65.6 | 1.008279798 |
| 60.44 | 0.169019597 | 62.72 | 0.907626866 |
| 57.24 | 0.155675945 | 59.94 | 0.821853064 |
| 54.06 | 0.141442716 | 57 | 0.729077319 |
| 51 | 0.132546947 | 54.12 | 0.660808374 |
| 47.94 | 0.120982449 | 51.08 | 0.594289915 |
| 44.74 | 0.112976257 | 48.12 | 0.541775343 |
| 41.42 | 0.104970066 | 45.12 | 0.496262713 |
| 38.08 | 0.096074297 | 42.04 | 0.448999597 |
| 34.84 | 0.088068106 | 39.06 | 0.410488911 |
| 31.6 | 0.084509799 | 35.96 | 0.371978224 |
| 28.4 | 0.076503607 | 32.76 | 0.333467537 |
| 25.16 | 0.070276569 | 29.66 | 0.299333065 |
| 21.92 | 0.064939108 | 26.5 | 0.266073835 |
| 18.42 | 0.058712071 | 23.26 | 0.23106412 |
| 15.18 | 0.054264187 | 20.06 | 0.195179162 |
| 12 | 0.045368418 | 16.86 | 0.161919933 |
| 8.84 | 0.040920534 | 13.58 | 0.130411189 |
| 5.66 | 0.031135189 | 10.28 | 0.099777688 |
| 2.02 | 0.013343652 | 7.04 | 0.066518459 |
| 0.58 | 0 | 3.88 | 0.035009715 |
| 0.44 | 0.00266873 | 1.24 | 0 |

Thermal Data

DSC was performed using a TA Instruments model 2920 module with a Thermal Analyst 5000 controller. Data were collected and analyzed using TA Instruments Thermal Solutions for NT Ver. 1.3L and Universal Analysis for NT Ver. 2.4F. Samples of about 1 mg were accurately weighed into coated aluminum pans with lids (TA part numbers 900779 and 900786), which were crimped to ensure good thermal contact pans with lids (TA part numbers 900796 and 900790). The samples were evaluated using a linear heating ramp of between 1° C./min and 10° C./min from rt to approximately 300° C. The cell was purged with a dry nitrogen flow of 50 standard cubic centimeters per minute (sccm).

The differential scanning calorimetry data was obtained for the anhydrous Crystal Form Ia of the mono-fumarate salt and hemi-fumarate salt of N-[(3R)-1-azabicyclo[2.2.2]oct-3-yl]furo[2,3-c]pyridine-5-carboxamide. Cystral Form Ia has a melting point at 195° C., while Crystal Form lb has a melting point at 238° C.

By the term “effective amount” of a compound as provided herein is meant a non-toxic but sufficient amount of the compound(s) to provide the desired effect. As pointed out below, the exact amount required will vary from subject to subject, depending on the species, age, and general condition of the subject, the severity of the disease that is being treated, the particular compound(s) used, the mode of administration, and the like. Thus, it is not possible to specify an exact “effective amount.” However, an appropriate effective amount may be determined by one of ordinary skill in the art using only routine experimentation.

The amount of therapeutically effective compound(s) that is administered and the dosage regimen for treating a disease condition with the compounds and/or compositions of this invention depends on a variety of factors, including the age, weight, sex and medical condition of the subject, the severity of the disease, the route and frequency of administration, and the particular compound(s) employed, and thus may vary widely. The compositions contain well know carriers and excipients in addition to a therapeutically effective amount of compounds of Formula I. The pharmaceutical compositions may contain active ingredient in the range of about 0.001 to 100 mg/kg/day for an adult, preferably in the range of about 0.1 to 50 mg/kg/day for an adult. A total daily dose of about 1 to 1000 mg of active ingredient may be appropriate for an adult. The daily dose can be administered in one to four doses per day.

In addition to the fumaric salt(s) of Formula I, the composition for therapeutic use may also comprise one or more non-toxic, pharmaceutically acceptable carrier materials or excipients. The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, and then tableted or encapsulated for convenient administration. Such capsules or tablets may contain a controlled-release formulation as may be provided in a dispersion of active compound in hydroxypropyl-methyl cellulose, or other methods known to those skilled in the art. For oral administration, the pharmaceutical composition may be in the form of, for example, a tablet, capsule, suspension or liquid. If desired, other active ingredients may be included in the composition.

In addition to the oral dosing, noted above, the compositions of the present invention may be administered by any suitable route, in the form of a pharmaceutical composition adapted to such a route, and in a dose effective for the treatment intended. The compositions may, for example, be administered parenterally, e.g., intravascularly, intraperitoneally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration.

The compounds may be dissolved in water, polyethylene glycol, propylene glycol, EtOH, corn oil, cottonseed oil, peanut oil, sesame oil, benzyl alcohol, sodium chloride, and/or various buffers. Other adjuvants and modes of administration are well and widely known in the pharmaceutical art.

The serotonin type 3 receptor (5HT₃R) is a member of a superfamily of ligand-gated ion channels, which includes the muscle and neuronal nAChR, the glycine receptor, and the γ-aminobutyric acid type A receptor. Like the other members of this receptor superfamily, the 5HT₃R exhibits a large degree of sequence homology with α7 nAChR but functionally the two ligand-gated ion channels are very different. For example, α7 nAChR is rapidly inactivated, is highly permeable to calcium and is activated by acetylcholine and nicotine. On the other hand, 5HT₃R is inactivated slowly, is relatively impermeable to calcium and is activated by serotonin. These experiments suggest that the α7 nAChR and 5HT₃R proteins have some degree of homology, but function very differently. Indeed the pharmacology of the channels is very different. For example, Ondansetron, a highly selective 5HT₃R antagonist, has little activity at the α7 nAChR. The converse is also true. For example, GTS-21, a highly selective α7 nAChR agonist, has little activity at the 5HT₃R. α7 nAChR is a ligand-gated Ca⁺⁺ channel formed by a homopentamer of α7 subunits. Previous studies have established that α-bungarotoxin (α-btx) binds selectively to this homopetameric, α7 nAChR subtype, and that α7 nAChR has a high affinity binding site for both α-btx and methyllycaconitine (MLA). α7 nAChR is expressed at high levels in the hippocampus, ventral tegmental area and ascending cholinergic projections from nucleus basilis to thalamocortical areas. α7 nAChR agonists increase neurotransmitter release, and increase cognition, arousal, attention, learning and memory.

Data from human and animal pharmacological studies establish that nicotinic cholinergic neuronal pathways control many important aspects of cognitive function including attention, learning and memory (Levin, E. D., Psychopharmacology, 108:417-31, 1992; Levin, E. D. and Simon B. B., Psychopharmacology, 138:217-30, 1998). For example, it is well known that nicotine increases cognition and attention in humans. ABT-418, a compound that activates α4β2 and α7 nAChR, improves cognition and attention in clinical trials of Alzheimer's disease and inattentive symptom cluster of ADHD (Potter, A. et. al., Psychopharmacology (Berl)., 142(4):334-42, March 1999; Wilens, T. E. et. al., Am. J. Psychiatry, 156(12):1931-7, December 1999). It is also clear that nicotine and selective but weak α7 nAChR agonists increase cognition and attention in rodents and non-human primates.

Schizophrenia is a complex multifactorial illness caused by genetic and non-genetic risk factors that produce a constellation of positive and negative symptoms. The positive symptoms include delusions and hallucinations and the negative symptoms include deficits in affect, attention, cognition and information processing. No single biological element has emerged as a dominant pathogenic factor in this disease. Indeed, it is likely that schizophrenia is a syndrome that is produced by the combination of many low penetrance risk factors. Pharmacological studies established that dopamine receptor antagonists are efficacious in treating the overt psychotic features (positive symptoms) of schizophrenia such as hallucinations and delusions. Clozapine, an “atypical” antipsychotic drug, is novel because it is effective in treating both the positive and some of the negative symptoms of this disease. Clozapine's utility as a drug is greatly limited because continued use leads to an increased risk of agranulocytosis and seizure. No other antipsychotic drug is effective in treating the negative symptoms of schizophrenia. This is significant because the restoration of cognitive functioning is the best predictor of a successful clinical and functional outcome of schizophrenic patients (Green, M. F., Am J Psychiatry, 153:321-30, 1996). By extension, it is clear that better drugs are needed to treat the cognitive disorders of schizophrenia in order to restore a better state of mental health to patients with this disorder.

One aspect of the cognitive deficit of schizophrenia can be measured by using the auditory event-related potential (P50) test of sensory gating. In this test, electroencepholographic (EEG) recordings of neuronal activity of the hippocampus are used to measure the subject's response to a series of auditory “clicks” (Adler, L. E. et. al., Biol. Psychiatry, 46:8-18, 1999). Normal individuals respond to the first click with greater degree than to the second click. In general, schizophrenics and schizotypal patients respond to both clicks nearly the same (Cullum, C. M. et. al., Schizophr. Res., 10:131-41, 1993). These data reflect a schizophrenic's inability to “filter” or ignore unimportant information. The sensory gating deficit appears to be one of the key pathological features of this disease (Cadenhead, K. S. et. al., Am. J. Psychiatry, 157:55-9, 2000). Multiple studies show that nicotine normalizes the sensory deficit of schizophrenia (Adler, L. E. et. al., Am. J. Psychiatry, 150:1856-61, 1993). Pharmacological studies indicate that nicotine's effect on sensory gating is via the α7 nAChR (Adler, L. E. et. al., Schizophr. Bull., 24:189-202, 1998). Indeed, the biochemical data indicate that schizophrenics have 50% fewer of α7 nAChR receptors in the hippocampus, thus giving a rationale to partial loss of α7 nAChR functionality (Freedman, R. et. al., Biol. Psychiatry, 38:22-33, 1995). Interestingly, genetic data indicate that a polymorphism in the promoter region of the α7 nAChR gene is strongly associated with the sensory gating deficit in schizophrenia (Freedman, R. et. al., Proc. Nat'l Acad. Sci. USA, 94(2):587-92, 1997; Myles-Worsley, M. et. al., Am. J. Med. Genet, 88(5):544-50, 1999). To date, no mutation in the coding region of the α7 nAChR has been identified. Thus, schizophrenics express the same α7 nAChR as non-schizophrenics.

Selective α7 nAChR agonists may be found using a functional assay on FLIPR (see WO 00/73431 A2). FLIPR is designed to read the fluorescent signal from each well of a 96 or 384 well plate as fast as twice a second for up to 30 minutes. This assay may be used to accurately measure the functional pharmacology of α7 nAChR and 5HT₃R. To conduct such an assay, one uses cell lines that expressed functional forms of the α7 nAChR using the α7/5-HT₃ channel as the drug target and cell lines that expressed functional 5HT₃R. In both cases, the ligand-gated ion channel was expressed in SH-EP1 cells. Both ion channels can produce robust signal in the FLIPR assay.

The compounds of the present invention are α7 nAChR agonists and may be used to treat a wide variety of diseases. For example, they may be used in treating schizophrenia, or psychosis.

Schizophrenia is a disease having multiple aspects. Currently available drugs are generally aimed at controlling the positive aspects of schizophrenia, such as delusions. One drug, Clozapine, is aimed at a broader spectrum of symptoms associated with schizophrenia. This drug has many side effects and is thus not suitable for many patients. Thus, there is a need for a drug to treat the cognitive and attention deficits associated with schizophrenia. Similarly, there is a need for a drug to treat the cognitive and attention deficits associated with schizoaffective disorders, or similar symptoms found in the relatives of schizophrenic patients.

Psychosis is a mental disorder characterized by gross impairment in the patient's perception of reality. The patient may suffer from delusions, and hallucinations, and may be incoherent in speech. His behavior may be agitated and is often incomprehensible to those around him. In the past, the term psychosis has been applied to many conditions that do not meet the stricter definition given above. For example, mood disorders were named as psychoses.

There are a variety of antipsychotic drugs. The conventional antipsychotic drugs include Chlorpromazine, Fluphenazine, Haloperidol, Loxapine, Mesoridazine, Molindone, Perphenazine, Pimozide, Thioridazine, Thiothixene, and Trifluoperazine. These drugs all have an affinity for the dopamine 2 receptor.

These conventional antipsychotic drugs have several side effects, including sedation, weight gain, tremors, elevated prolactin levels, akathisia (motor restlessness), dystonia and muscle stiffness. These drugs may also cause tardive dyskinesia. Unfortunately, only about 70% of patients with schizophrenia respond to conventional antipsychotic drugs. For these patients, atypical antipsychotic drugs are available.

Atypical antipsychotic drugs generally are able to alleviate positive symptoms of psychosis while also improving negative symptoms of the psychosis to a greater degree than conventional antipsychotics. These drugs may improve neurocognitive deficits. Extrapyramidal (motor) side effects are not as likely to occur with the atypical antipsychotic drugs, and thus, these atypical antipsychotic drugs have a lower risk of producing tardive dyskinesia. Finally these atypical antipsychotic drugs cause little or no elevation of prolactin. Unfortunately, these drugs are not free of side effects. Although these drugs each produce different side effects, as a group the side effects include: agranulocytosis; increased risk of seizures, weight gain, somnolence, dizziness, tachycardia, decreased ejaculatory volume, and mild prolongation of QTc interval.

In a combination therapy to treat multiple symptoms of diseases such as schizophrenia, the compounds of Formula I and the anti-psychotic drugs can be administered simultaneously or at separate intervals. When administered simultaneously the compounds of Formula I and the anti-psychotic drugs can be incorporated into a single pharmaceutical composition, e.g., a pharmaceutical combination therapy composition. Alternatively, two separate compositions, i.e., one containing compounds of Formula I and the other containing anti-psychotic drugs, can be administered simultaneously. Examples of anti-psychotic drugs, in addition to those listed above, include, but are not limited to, Thorazine, Mellaril, Trilafon, Navane, Stelazine, Permitil, Prolixin, Risperdal, Zyprexa, Seroquel, ZELDOX, Acetophenazine, Carphenazine, Chlorprothixene, Droperidol, Loxapine, Mesoridazine, Molindone, Ondansetron, Pimozide, Prochlorperazine, and Promazine.

A pharmaceutical combination therapy composition can include therapeutically effective amounts of the compounds of Formula I, noted above, and a therapeutically effective amount of anti-psychotic drugs. These compositions may be formulated with common excipients, diluents or carriers, and compressed into tablets, or formulated elixirs or solutions for convenient oral administration or administered by intramuscular intravenous routes. The compounds can be administered rectally, topically, orally, sublingually, or parenterally and may be formulated as sustained relief dosage forms and the like.

When separately administered, therapeutically effective amounts of compositions containing compounds of Formula I and anti-psychotic drugs are administered on a different schedule. One may be administered before the other as long as the time between the two administrations falls within a therapeutically effective interval. A therapeutically effective interval is a period of time beginning when one of either (a) the compounds of Formula I, or (b) the anti-psychotic drugs is administered to a human and ending at the limit of the beneficial effect in the treatment of schizophrenia or psychosis of the combination of (a) and (b). The methods of administration of the compounds of Formula I and the anti-psychotic drugs may vary. Thus, either agent or both agents may be administered rectally, topically, orally, sublingually, or parenterally.

As discussed, the compounds of the present invention are α7 nAChR agonists. Therefore, as another aspect of the present invention, the compounds of the present invention may be used to treat a variety of diseases including cognitive and attention deficit symptoms of Alzheimer's, neurodegeneration associated with diseases such as Alzheimer's disease, pre-senile dementia (also known as mild cognitive impairment), and senile dementia.

Alzheimer's disease has many aspects, including cognitive and attention deficits. Currently, these deficits are treated with cholinesterase inhibitors. These inhibitors slow the break down of acetylcholine, and thereby provide a general nonspecific increase in the activity of the cholinergic nervous system. Since the drugs are nonspecific, they have a wide variety of side effects. Thus, there is a need for a drug that stimulates a portion of the cholinergic pathways and thereby provides improvement in the cognitive and attention deficits associated with Alzheimer's disease without the side effects created by nonspecific stimulation of the cholinergic pathways.

Neurodegeneration is a common problem associated with diseases such as Alzheimer's disease. While the current drugs treat some of the symptoms of this disease, they do not control the underlying pathology of the disease. Accordingly, it would be desirable to provide a drug that can slow the progress of Alzheimer's disease.

Pre-senile dementia (mild cognitive impairment) concerns memory impairment rather than attention deficit problems and otherwise unimpaired cognitive functioning. Mild cognitive impairment is distinguished from senile dementia in that mild cognitive impairment involves a more persistent and troublesome problem of memory loss for the age of the patient. There currently is no medication specifically identified for treatment of mild cognitive impairment, due somewhat to the newness of identifying the disease. Therefore, there is a need for a drug to treat the memory problems associated with mild cognitive impairment.

Senile dementia is not a single disease state. However, the conditions classified under this name frequently include cognitive and attention deficits. Generally, these deficits are not treated. Accordingly, there is a need for a drug that provides improvement in the cognitive and attention deficits associated with senile dementia.

As discussed, the compounds of the present invention are α7 nAChR agonists. Therefore, yet other diseases to be treated with compounds of the present invention include treating the cognitive and attention deficits as well as the neurodegeneration associated with any one or more or combination of the following: attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, Parkinson's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, or symptoms associated with pain.

Attention deficit disorder is generally treated with methylphenidate, an amphetamine-like molecule that has some potential for abuse. Accordingly, it would be desirable to provide a drug that treats attention deficit disorder while having fewer side effects than the currently used drug.

Attention deficit hyperactivity disorder, otherwise known as ADHD, is a neurobehavioral disorder affecting 3-5% of all American children. ADHD concerns cognitive alone or both cognitive and behavioral actions by interfering with a person's ability to stay on a task and to exercise age-appropriate inhibition. Several types of ADHD exist: a predominantly inattentive subtype, a predominantly hyperactive-impulsive subtype, and a combined subtype. Treatment may include medications such as methylphenidate, dextroamphetamine, or pemoline, which act to decrease impulsivity and hyperactivity and to increase attention. No “cure” for ADHD currently exists. Children with the disorder seldom outgrow it; therefore, there is a need for appropriate medicaments.

Depression is a mood disorder of varying lengths of normally several months to more than two years and of varying degrees of feelings involving sadness, despair, and discouragement. The heterocyclic antidepressants (HCA's) are currently the largest class of antidepressants, but monoamine oxidase inhibitors (MAOI's) are used in particular types of depression. Common side effects from HCA's are sedation and weight gain. In elderly patients with organic brain disease, the side effects from HCA's can also include seizures and behavioral symptoms. The main side effects from using MAOI's occur from dietary and drug interactions. Therefore, agents with fewer side effects would be useful.

Anxiety disorders (disorders with prominent anxiety or phobic avoidance), represent an area of umet medical needs in the treatment of psychiatric illness. See Diagnostic & Statistical Manual of Mental Disorders, IV (1994), pp 393-394, for various disease forms of anxiety.

General anxiety disorder (GAD) occurs when a person worries about things such as family, health, or work when there is no reason to worry and is unable not to worry. About 3 to 4% of the U.S. population has GAD during the course of a year. GAD most often strikes people in childhood or adolescence, but can begin in adulthood, too. It affects women more often than men. Currently, treatment involves cognitive-behavioral therapy, relaxation techniques, and biofeedback to control muscle tension and medications such as benzodiazepines, imipramine, and buspirone. These drugs are effective but all have side-effect liabilities. Therefore, there is a need of a pharmaceutical agent to address the symptoms with fewer side effects.

Anxiety also includes post-traumatic stress disorder (PTSD), which is a form of anxiety triggered by memories of a traumatic event that directly affected the patient or that the patient may have witnessed. The disorder commonly affects survivors of traumatic events including sexual assault, physical assault, war, torture, natural disasters, an automobile accident, an airplane crash, a hostage situation, or a death camp. The affliction also can affect rescue workers at an airplane crash or a mass shooting, someone who witnessed a tragic accident or someone who has unexpectedly lost a loved one. Treatment for PTSD includes cognitive-behavioral therapy, group psychotherapy, and medications such as Clonazepam, Lorazepam and selective serotonin-reuptake inhibitors such as Fluoxetine, Sertraline, Paroxetine, Citalopram and Fluvoxamine. These medications help control anxiety as well as depression. Various forms of exposure therapy (such as systemic desensitization and imaginal flooding) have all been used with PTSD patients. Exposure treatment for PTSD involves repeated reliving of the trauma, under controlled conditions, with the aim of facilitating the processing of the trauma. Therefore, there is a need for better pharmaceutical agents to treat post traumatic stress disorder.

Mood and affective disorders fall within a large group of diseases, including monopolar depression and bi-polar mood disorder. These diseases are treated with three major classes of compounds. The first group is the heterocyclic antidepressant (HCA's). This group includes the well-known tricyclic antidepressants. The second group of compounds used to treat mood disorders is the monoamine oxidase inhibitors (MAOI's) that are used in particular types of diseases. The third drug is lithium. Common side effects from HCA's are sedation and weight gain. In elderly patients with organic brain disease, the side effects of HCA's can also include seizures and behavioral symptoms. The main side effects from using MAOI's occur from dietary and drug interactions. Benign side effects from the use of lithium include, but are not limited to, weight gain, nausea, diarrhea, polyuria, polydipsia, and tremor. Toxic side effects from lithium can include persistent headache, mental confusion, and may reach seizures and cardiac arrhythmias. Therefore, agents with less side effects or interactions with food or other medications would be useful.

Borderline personality disorder, although not as well known as bipolar disorder, is more common. People having borderline personality disorder suffer from a disorder of emotion regulation. Pharmaceutical agents are used to treat specific symptoms, such as depression or thinking distortions.

Acquired immune deficiency syndrome (AIDS) results from an infection with the human immunodeficiency virus (HIV). This virus attacks selected cells and impairs the proper function of the immune, nervous, and other systems. HIV infection can cause other problems such as, but not limited to, difficulties in thinking, otherwise known as AIDS dementia complex. Therefore, there is a need to drugs to relieve the confusion and mental decline of persons with AIDS.

Amyotrophic lateral sclerosis, also known as Lou Gehrig's disease, belongs to a class of disorders known as motor neuron diseases wherein specific nerve cells in the brain and spinal cord gradually degenerate to negatively affect the control of voluntary movement. Currently, there is no cure for amyotrophic lateral sclerosis although patients may receive treatment from some of their symptoms and although Riluzole has been shown to prolong the survival of patients. Therefore, there is a need for a pharmaceutical agent to treat this disease.

Traumatic brain injury occurs when the brain is damaged from a sudden physical assault on the head. Symptoms of the traumatic brain injury include confusion and other cognitive problems. Therefore, there is a need to address the symptoms of confusion and other cognitive problems.

Brain tumors are abnormal growths of tissue found inside of the skull. Symptoms of brain tumors include behavioral and cognitive problems. Surgery, radiation, and chemotherapy are used to treat the tumor, but other agents are necessary to address associated symptoms. Therefore, there is a need to address the symptoms of behavioral and cognitive problems.

Persons with Down's syndrome have in all or at least some of their cells an extra, critical portion of the number 21 chromosome. Adults who have Down's syndrome are known to be at risk for Alzheimer-type dementia. Currently, there is no proven treatment for Down's syndrome. Therefore, there is a need to address the dementia associated with Down's syndrome.

Genetically programmed degeneration of neurons in certain areas of the brain cause Huntington's disease. Early symptoms of Huntington's disease include mood swings, or trouble learning new things or remembering a fact. Most drugs used to treat the symptoms of Huntington's disease have side effects such as fatigue, restlessness, or hyperexcitability. Currently, there is no treatment to stop or reverse the progression of Huntington's disease. Therefore, there is a need of a pharmaceutical agent to address the symptoms with fewer side effects.

Dementia with Lewy Bodies is a neurodegenerative disorder involving abnormal structures known as Lewy bodies found in certain areas of the brain. Symptoms of dementia with Lewy bodies include, but are not limited to, fluctuating cognitive impairment with episodic delirium. Currently, treatment concerns addressing the parkinsonian and psychiatric symptoms. However, medicine to control tremors or loss of muscle movement may actually accentuate the underlying disease of dementia with Lewy bodies. Therefore, there is a need of a pharmaceutical agent to treat dementia with Lewy bodies.

Parkinson's disease is a neurological disorder characterized by tremor, hypokinesia, and muscular rigidity. Currently, there is no treatment to stop the progression of the disease. Therefore, there is a need of a pharmaceutical agent to address Parkinson's.

Tardive dyskinesia is associated with the use of conventional antipsychotic drugs. This disease is characterized by involuntary movements most often manifested by puckering of the lips and tongue and/or writhing of the arms or legs. The incidence of tardive dyskinesia is about 5% per year of drug exposure among patients taking conventional antipsychotic drugs. In about 2% of persons with the disease, tardive dyskinesia is severely disfiguring. Currently, there is no generalized treatment for tardive dyskinesia. Furthermore, the removal of the effect-causing drugs is not always an option due to underlying problems. Therefore, there is a need for a pharmaceutical agent to address the symptoms of tardive dyskinesia.

Pick's disease results from a slowly progressive deterioration of social skills and changes in personality with the resulting symptoms being impairment of intellect, memory, and language. Common symptoms include memory loss, lack of spontaneity, difficulty in thinking or concentrating, and speech disturbances. Currently, there is no specific treatment or cure for Pick's disease but some symptoms can be treated with cholinergic and serotonin-boosting antidepressants. In addition, antipsychotic medications may alleviate symptoms in FTD patients who are experiencing delusions or hallucinations. Therefore, there is a need for a pharmaceutical agent to treat the progressive deterioration of social skills and changes in personality and to address the symptoms with fewer side effects.

Dysregulation of food intake associated with eating disease, including bulemia nervosa and anorexia nervosa, involve neurophysiological pathways. Anorexia nervosa is hard to treat due to patients not entering or remaining in after entering programs. Currently, there is no effective treatment for persons suffering from severe anorexia nervosa. Cognitive behavioral therapy has helped patients suffering from bulemia nervosa; however, the response rate is only about 50% and current treatment does not adequately address emotional regulation. Therefore, there is a need for pharmaceutical agents to address neurophysiological problems underlying diseases of dysregulation of food intake.

Cigarette smoking has been recognized as a major public health problem for a long time. However, in spite of the public awareness of health hazard, the smoking habit remains extraordinarily persistent and difficult to break. There are many treatment methods available, and yet people continue to smoke. Administration of nicotine transdermally, or in a chewing gum base is common treatments. However, nicotine has a large number of actions in the body, and thus can have many side effects. It is clear that there is both a need and a demand of long standing for a convenient and relatively easy method for aiding smokers in reducing or eliminating cigarette consumption. A drug that could selectively stimulate only certain of the nicotinic receptors would be useful in smoke cessation programs.

Smoke cessation programs may involve oral dosing of the drug of choice. The drug may be in the form of tablets. However, it is preferred to administer the daily dose over the waking hours, by administration of a series of incremental doses during the day. The preferred method of such administration is a slowly dissolving lozenge, troche, or chewing gum, in which the drug is dispersed. Another drug in treating nicotine addiction is Zyban. This is not a nicotine replacement, as are the gum and patch. Rather, this works on other areas of the brain, and its effectiveness is to help control nicotine craving or thoughts about cigarette use in people trying to quit. Zyban is not very effective and effective drugs are needed to assist smokers in their desire to stop smoking. These drugs may be administered transdermally through the use of skin patches. In certain cases, the drugs may be administered by subcutaneous injection, especially if sustained release formulations are used.

Drug use and dependence is a complex phenomenon, which cannot be encapsulated within a single definition. Different drugs have different effects, and therefore different types of dependence. Drug dependence has two basic causes, that is, tolerance and physical dependence. Tolerance exists when the user must take progressively larger doses to produce the effect originally achieved with smaller doses. Physical dependence exists when the user has developed a state of physiologic adaptation to a drug, and there is a withdrawal (abstinence) syndrome when the drug is no longer taken. A withdrawal syndrome can occur either when the drug is discontinued or when an antagonist displaces the drug from its binding site on cell receptors, thereby counteracting its effect. Drug dependence does not always require physical dependence.

In addition drug dependence often involves psychological dependence, that is, a feeling of pleasure or satisfaction when taking the drug. These feelings lead the user to repeat the drug experience or to avoid the displeasure of being deprived of the drug. Drugs that produce strong physical dependence, such as nicotine, heroin and alcohol are often abused, and the pattern of dependence is difficult to break. Drugs that produce dependence act on the CNS and generally reduce anxiety and tension; produce elation, euphoria, or other pleasurable mood changes; provide the user feelings of increased mental and physical ability; or alter sensory perception in some pleasurable manner. Among the drugs that are commonly abused are ethyl alcohol, opioids, anxiolytics, hypnotics, cannabis (marijuana), cocaine, amphetamines, and hallucinogens. The current treatment for drug-addicted people often involves a combination of behavioral therapies and medications. Medications, such as methadone or LAAM (levo-alpha-acetyl-methadol), are effective in suppressing the withdrawal symptoms and drug craving associated with narcotic addiction, thus reducing illicit drug use and improving the chances of the individual remaining in treatment. The primary medically assisted withdrawal method for narcotic addiction is to switch the patient to a comparable drug that produces milder withdrawal symptoms, and then gradually taper off the substitute medication. The medication used most often is methadone, taken orally once a day. Patients are started on the lowest dose that prevents the more severe signs of withdrawal and then the dose is gradually reduced. Substitutes can be used also for withdrawal from sedatives. Patients can be switched to long-acting sedatives, such as diazepam or phenobarbital, which are then gradually reduced.

Gilles de la Tourette's Syndrome is an inherited neurological disorder. The disorder is characterized by uncontrollable vocal sounds called tics and involuntary movements. The symptoms generally manifest in an individual before the person is 18 years of age. The movement disorder may begin with simple tics that progress to multiple complex tics, including respiratory and vocal ones. Vocal tics may begin as grunting or barking noises and evolve into compulsive utterances. Coprolalia (involuntary scatologic utterances) occurs in 50% of patients. Severe tics and coprolalia may be physically and socially disabling. Tics tend to be more complex than myoclonus, but less flowing than choreic movements, from which they must be differentiated. The patient may voluntarily suppress them for seconds or minutes.

Currently simple tics are often treated with benzodiazepines. For simple and complex tics, Clonidine may be used. Long-term use of Clonidine does not cause tardive dyskinesia; its limiting adverse effect is hypotension. In more severe cases, antipsychotics, such as Haloperidol may be required, but side effects of dysphoria, parkinsonism, akathisia, and tardive dyskinesia may limit use of such antipsychotics. There is a need for safe and effective methods for treating this syndrome.

Age-related macular degeneration (AMD) is a common eye disease of the macula which is a tiny area in the retina that helps produce sharp, central vision required for “straight ahead” activities that include reading and driving. Persons with AMD lose their clear, central vision. AMD takes two forms: wet and dry. In dry AMD, there is a slow breakdown of light-sensing cells in the macula. There currently is no cure for dry AMD. In wet AMD, new, fragile blood vessels growing beneath the macula as dry AMD worsens and these vessels often leak blood and fluid to cause rapid damage to the macula quickly leading to the loss of central vision. Laser surgery can treat some cases of wet AMD. Therefore, there is a need of a pharmaceutical agent to address AMD.

Glaucoma is within a group of diseases occurs from an increase in intraocular pressure causing pathological changes in the optical disk and negatively affects the field of vision. Medicaments to treat glaucoma either decrease the amount of fluid entering the eye or increase drainage of fluids from the eye in order to decrease intraocular pressure. However, current drugs have drawbacks such as not working over time or causing side effects so the eye-care professional has to either prescribe other drugs or modify the prescription of the drug being used. There is a need for safe and effective methods for treating problems manifesting into glaucoma.

Ischemic periods in glaucoma cause release of excitotoxic amino acids and stimulate inducible form of nitric oxide synthase (iNOS) leading to neurodegeneration. Alpha 7 nicotinic agonists may stimulate the release of inhibitory amino acids such as GABA which will dampen hyperexcitablity. Alpha 7 nicotinic agonists are also directly neuroprotective on neuronal cell bodies. Thus, alpha 7 nicotinic agonists have the potential to be neuroprotective in glaucoma.

Persons afflicted with pain often have what is referred to as the “terrible triad” of suffering from the pain, resulting in sleeplessness and sadness, all of which are hard on the afflicted individual and that individual's family. Pain can manifest itself in various forms, including, but not limited to, headaches of all severity, back pain, neurogenic, and pain from other ailments such as arthritis and cancer from its existence or from therapy to irradicate it. Pain can be either chronic (persistent pain for months or years) or acute (short-lived, immediate pain to inform the person of possible injury and need of treatment). Persons suffering from pain respond differently to individual therapies with varying degrees of success. There is a need for safe and effective methods for treating pain.

Finally, the compounds of the present invention may be used in combination therapy with typical and atypical anti-psychotic drugs (also called an anti-psychotic agent). All compounds within the present invention are useful for and may also be used in combination with each other to prepare pharmaceutical compositions. Such combination therapy lowers the effective dose of the anti-psychotic drug and thereby reduces the side effects of the anti-psychotic drugs. Some typical anti-psychotic drugs that may be used in the practice of the invention include Haldol. Some atypical anti-psychotic drugs include Ziprasidone, Olanzapine, Resperidone, and Quetiapine.

### EXAMPLE 1

### N-[(3R)-1-azabicyclo[2.2.2]oct-3-yl]furo[2,3-c]pyridine-5-carboxamide

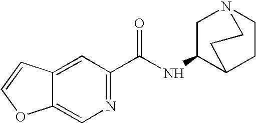

Example 1 is obtained by coupling furo[2,3-c]pyridine-5-carboxylic acid with R-(+)-3-aminoquinuclidine. There are many routes for obtaining the carboxylic acid including the preparation of the acid discussed herein and also from hydrolyzing the ester, the preparation of which is discussed in U.S. Pat. No. 6,265,580. n-Butyl furo[2,3-c]pyridine-5-carboxylate is hydrolyzed to the corresponding carboxylate salt on treatment with sodium or potassium hydroxide in aqueous methanol or acetonitrile-methanol mixtures. Acidification to pH 2.5-3.5 generates the carboxylic acid, which is isolated as a solid. The free base can also be prepared directly from n-butyl furo[2,3-c]pyridine-5-carboxylate by direct condensation using at least 1.5 molar equivalents of (R)-3-aminoquinuclidine and heating in ethanol or n-butyl alcohol.

2-Chloro-3-pyridinol (20.0 g, 0.154 mol), NaHCO₃ (19.5g, 0.232 mol, 1.5 equ), and 150 mL of water are placed in a flask. The flask is placed in an oil bath at 90° C., and after 5 min, 37% aqueous formaldehyde (40.5 mL, 0.541 mol, 3.5 equ) is added in six unequal doses in the following order: 12 mL, 3×8 mL, then 2.2 mL all at 90-minute intervals and then the final 2.3 mL after the reaction had stirred for 15 h at 90° C. The reaction is stirred at 90° C. for another 4 h and then is cooled by placing the flask in an ice bath. The pH of the reaction is then adjusted to 1 using 6N HCl. The reaction is stirred for 1.5 h in an ice bath allowing an undesired solid to form. The undesired solid is removed by filtration, and the filtrate is extracted seven times with EtOAc. The combined organic extracts are concentrated in vacuo, toluene is added to the flask and removed in vacuo to azeotrope water, and then CH₂Cl₂ is added and removed in vacuo to obtain 2-chloro-6-(hydroxymethyl)-3-pyridinol (C1) as a pale yellow solid (81% yield) sufficiently pure for subsequent reaction. MS (EI) for C₆H₆ClNO₂, m/z: 159(M)⁺.

C1 (11.6 g, 72.7 mmol) and NaHCO₃ (18.3 g, 218 mmol) are added to 200 mL water. The mixture is stirred until homogeneous, the flask is placed in an ice bath, iodine (19.4 g, 76.3 mmol) is added, and the reaction is stirred over the weekend at rt. The pH of the mixture is adjusted to 3 with 2N NaHSO₄, and the mixture is extracted with 4×50 mL EtOAc. The combined organic layer is dried (MgSO₄), is filtered, and the filtrate is concentrated in vacuo to a yellow solid. The crude solid is washed with EtOAc to provide 2-chloro-6-(hydroxymethyl)-4-iodo-3-pyridinol (C2) as an off-white solid (62% yield), and the filtrate is concentrated to a small volume and is chromatographed over 250 g silica gel (230-400 mesh) eluting with 2.5:4.5:4:0.1 EtOAc/CH₂Cl₂/hexane/acetic acid. The fractions with the desired compound are combined and concentrated to afford additional pure C2 (12% yield). MS (EI) for C₆H₅ClINO₂, m/z: 285(M)⁺.

C2 (13.9 g, 48.6 mmol) is combined with trimethylsilylacetylene (9.6 mL, 68 mmol), bis(triphenylphosphine)palladium dichloride (1.02 g, 1.46 mmol) and cuprous iodide (139 mg, 0.73 mmol) in 80 mL CHCl₃/40 mL THF under N₂. TEA (21 mL, 151 mmol) is added, and the reaction is stirred 3 h at rt and is diluted with 200 mL CHCl₃. The mixture is washed with 2×150 mL 5% HCl and the combined aqueous layers are extracted with 2×50 mL CHCl₃. The combined organic layer is washed with 100 mL 50% saturated NaCl, is dried (MgSO₄), and is concentrated in vacuo to an amber oil. The crude material is chromatographed over 350 g silica gel (230-400 mesh), eluting with 35% EtOAc/hexane. The fractions with the desired compound are combined and concentrated to afford 2-chloro-6-(hydroxymethyl)-4-[(trimethylsilyl)ethynyl]-3-pyridinol (C3) as a golden solid (92% yield). MS (EI) for C₁₁H₁₄ClNO₂Si, m/z: 255(M)⁺.

C3 (7.9 g, 31.2 mmol) and cuprous iodide (297 mg, 1.6 mmol) in 60 mL EtOH/60 mL TEA are added to a flask. The reaction is placed in an oil bath at 70° C. for 3.5 h, is cooled to rt, and concentrated in vacuo. The residue is partitioned between 100 mL 5% HCl and CH₂Cl₂ (4×50 mL). The combined organic layer is dried (MgSO₄), filtered, and concentrated in vacuo to give 6.5 g of a crude amber solid. The crude material is chromatographed over 300 g silica gel (230-400 mesh) eluting with 30-40% EtOAc/hexane. Two sets of fractions with two different desired compounds are identified by TLC/UV. The two compounds eluted separately. The early-eluting pool of fractions is combined and concentrated to afford [7-chloro-2-(trimethylsilyl)furo[2,3-c]pyridin-5-yl]methanol (C5) as a white solid (46% yield). The later-eluting pool of fractions is combined and concentrated to provide (7-chlorofuro[2,3-c]pyridin-5-yl)methanol (4) as a white solid (27% yield). MS (EI) for C₈H₆ClNO₂, m/z: 183 (M)+for C4. HRMS (FAB) calculated for C₁₁H₁₄ClNO₂Si m/z: 255.0482, found 255.0481 for C5.

C5 (1.05 g, 4.1 mmol) and 10% Pd/C catalyst (1.05 g) are placed in 20 mL absolute EtOH. Cyclohexene (4 mL, 40.1 mmol) is added, and the reaction is refluxed for 2.5 h, and then filtered through celite. The filter cake is washed with 1:1 EtOH/CH₂Cl₂, and the filtrate is concentrated to a pale yellow solid. The residue is partitioned between 40 mL saturated NaHCO₃ and extracted with CH₂Cl₂ (4×20 mL). The combined organic layer is dried (MgSO₄), filtered, and concentrated in vacuo to a pale oil (1.04 g). The pale oil is chromatographed over 50 g silica gel (230-400 mesh) eluting with 50-70% EtOAc/hexane. The fractions with the desired compound are combined and concentrated to afford 5-hydroxymethyl-2-trimethylsilyl-furo[2,3-c]pyridine (C6) as a white solid (90% yield). MS (EI) for C₁₁H₁₅NO₂Si, m/z: 221(M)⁺.

C6 (770 mg, 3.48 mmol) is dissolved in 10 mL MeOH. 2N NaOH (3 mL, 6 mmol) is added, and the reaction is stirred for 1.5 h at rt. The solution is concentrated in vacuo to a residue. Water (20 mL) is added to the residue and extracted with 4×10 mL CH₂Cl₂. The combined organic layer is dried (K₂CO₃), filtered, and then concentrated in vacuo to afford furo[2,3-c]pyridin-5-yl methanol (C7) as a white solid (90% yield). Analysis calculated for C₈H₇NO₂: C, 64.42; H, 4.73; N, 9.39. Found: C, 64.60; H, 4.56; N, 9.44.

Alternatively, C3 is used to obtain C7 with fewer steps: C3 (44.6 g, 174.4 mmol) is combined with cuprous iodide (1.66 g, 8.72 mmol) and diisopropylamine (44 ml, 300 mmol) in 300 ml methanol under nitrogen. The reaction is warmed to 45-50° C. for 6 h, is cooled to rt and treated with 100 ml saturated NaHCO₃ followed by 100 ml 2N NaOH. The dark mixture is stirred overnight, filtered through celite, the volatiles removed in vacuo and the residue is partitioned between 1×500 ml water and 4×200 ml CH₂Cl₂ (some filtrations is required to effect good separation). The combined organic layer is dried (MgSO₄) and concentrated in vacuo to afford C4 (25.25 g, 79%) as a pale orange solid. Anal. Calcd for C₈H₆ClNO₂: C, 52.34; H, 3.29; N, 7.63. Found: C, 52.27; H, 3.23; N, 7.57.

C4 (32.0 g, 174 mmol) is combined with zinc powder (34.2 g, 523 mmol) in absolute EtOH (900 mL), using an overhead stirrer. The mixture is heated to 70° C., HCl (87.2 mL, 1.05 mol) is added slowly drop-wise, and the mixture is heated to reflux for 1 h. The mixture is cooled slightly, filtered to remove the metallic zinc and concentrated to near-dryness. The yellow oil is diluted with H₂O (150 mL) and EtOAc (950 mL) and is treated slowly drop-wise with 20% Na₂CO₃ (310 mL) as the mixture is warmed to reflux. The vigorously stirred (using overhead stirrer) mixture is refluxed for 1 h, cooled slightly and the organics removed via cannula under reduced pressure. Additional EtOAc (600 mL) is added, the mixture is heated to reflux for 1 h, cooled slightly and the organics removed as above. More EtoAc (600 mL) is added, the mixture is stirred at rt overnight then heated to reflux for 1 h, cooled slightly and most of the organics removed as above. The remaining mixture is filtered through celite, rinsed with EtOAc until no additional product elutes, and the layers separated. The aqueous layer is further extracted with EtOAc (2×400 mL). The combined organics are dried (MgSO₄) and concentrated to a dark yellow solid (23.6 g). The crude material is chromatographed over 900 g slurry-packed silica gel, eluting with 60% EtOAc/hexane (3 L), 70% EtOAc/hexane (2 L), and finally 100% EtOAc. The appropriate fractions are combined and concentrated in vacuo to afford C7 (19.5 g, 75%) as a white solid. Anal. Calcd for C₈H₇NO₂: C, 64.42; H, 4.73; N, 9.39; Found: C, 64.60; H, 4.56; N, 9.44.

Oxalyl chloride (685 μL, 7.8 mmol) is dissolved in 30 mL CH₂Cl₂ in a dry flask under N₂. The flask is placed in a dry-ice/acetone bath, DMSO (1.11 mL, 15.6 mmol) in 5 mL CH₂Cl₂ is added drop-wise, and the mixture is stirred for 20 min. C7 (1.0 g, 6.7 mmol) in 10 mL CH₂Cl₂ is added, and the reaction is stirred 30 min at −78° C. TEA (4.7 mL, 33.5 mmol) is added, the reaction is allowed to warm to rt, is stirred 1 h, and is washed with 25 mL saturated NaHCO₃. The organic layer is dried (K₂CO₃), filtered, and concentrated in vacuo to an orange solid. The crude material is chromatographed over 50 g silica gel (230-400 mesh) eluting with 33% EtOAc/hexane. The fractions with the desired compound are combined and concentrated to provide furo[2,3-c]pyridine-5-carbaldehyde (C8) as a white solid (86% yield). MS (EI) for C₈H₅NO₂, m/z: 147 (M)⁺.

C8 (850 mg, 5.8 mmol) is dissolved in 10 mL DMSO. KH₂PO₄ (221 mg, 1.6 mmol) in 3 mL water is added and then NaClO₂ (920 mg, 8.2 mmol) in 7 mL water is added, and the reaction is stirred 3h at rt. The reaction is diluted with 25 mL water, the pH is adjusted to 10 with 2N NaOH, and the mixture is extracted with 3×20 mL ether. The combined ether layer is discarded. The pH of the aqueous layer is adjusted to 3.5 with 10% aqueous HCl and is extracted with 13×10 mL 10% MeOH/CH₂Cl₂. The MeOH/CH₂Cl₂ organic layer is dried (Na₂SO₄), filtered, and concentrated in vacuo to a pale oil. The residual DMSO is removed under a stream of N₂ at rt to provide a white paste. The paste is dissolved in MeOH and is concentrated to dryness. The white solid is washed with ether and dried to afford crude furo[2,3-c]pyridine-5-carboxylic acid (C9) (94% yield). MS (ESI) for C₈H₅NO₃, 162.8 (M−H)⁻.

Acid C9 (1.96 g, 12.0 mmol), DIEA (6.27 mL, 36.0 mmol), and R-(+)-3-aminoquinuclidine dihydrochloride (2.42 g, 12.1 mmol) are added to DMF (60 mL), and the reaction is cooled in an ice bath. HATU (4.57 g, 12.0 mmol) is added, the solution allowed to warm to rt over 2.5 h, then concentrated in vacuo. The residue is stirred with saturated NaHCO₃ (30 mL) for 30 min, then extracted with CHCl₃ (10×50 mL). The combined organic layer is dried (Na₂SO₄) and is concentrated in vacuo. The crude material is chromatographed over 130 g slurry-packed silica gel, eluting with 0.5% ammonium hydroxide in 10% MeOH/CHCl₃. The appropriate fractions are combined and concentrated to a residue.

Salt Formation:

Mono Fumarate Salt:

### EXAMPLE 1(a)(i)

The free base (556 mg, 2.05 mmol) is dissolved in 4 ml isopropanol. Fumaric acid (238 mg, 2.05 mmol) is dissolved in 0.5 ml MeOH, the solution is diluted with 5 ml acetone, and the mixture is added in one addition to the solution of free-base. The reaction is stirred 2h, the thick slurry is diluted with 10 ml acetone, and the mixture is stirred overnight. The solid is collected, washed with fresh acetone, and dried to afford 680 mg (86%) of Example 1(a)(i). ¹H NMR (300 MHz, DMSO-d₆) δ 1.64, 1.85, 2.00, 2.11, 3.07, 3.25, 3.50, 4.32, 6.48, 7.21, 8.35, 8.41, 9.05 ppm. Anal. Calcd for C₁₉H₂₁N₃O₆: C, 58.91; H, 5.46; N, 10.85. Found: C, 58.78; H, 5.50; N, 10.79.

### EXAMPLE 1(a)(ii)

Free base (20.5 g) and fumaric acid (8.93 g) are combined with n-butyl alcohol (540 mL) and water (22 mL). The mixture is stirred and heated to between 70-80° C. to produce a solution that is clarified by filtration. The clarified solution is cooled to between 25° C.-30° C. and then concentrated by distillation under vacuum to about 330 mL volume to precipitate Example 1(a)(ii). The slurry is stirred at 70° C.-80° C. for 14 hours and then cooled to 23° C. After 1 hour additional stirring, Example 1(a)(ii) is collected by filtration and washed with two 50 ml portions of n-butyl alcohol. Example 1(a)(ii) is dried with ambient nitrogen flow and then under vacuum at 60° C. to provide 25.4 g of Example I (a)(ii) (87%).

Hemi Fumarate Salt:

### EXAMPLE 1(b)

Fumaric acid (437 mg) is dissolved in 15 grams of IPA by heating to a jacket temperature of about 75° C. (reactor temperature about 72° C.) in a 100 ml jacketed reactor. In a separate reactor, the free base (19.98 grams as a 10% by weight/weight in IPA) solution is heated to the jacket temperature of about 75° C. Stirring is set at about 145 rpm in both the reactors. Once all of the fumaric acid dissolves, this solution is transferred to the free-base solution through a transfer pipette, while maintaining the temperature at 72° C. The transfer is complete within 10 minutes. Solids started precipitating out towards the end of the transfer. The slurry is held at 75° C. for 1 hour and cooled to 20° C. in ten hours using a linear cooling ramp. It is held at 20° C. for seven hours and then discharged on a 60 ml medium frit sintered glass funnel. The cake is washed with IPA (5 ml) and air dried for 15 minutes. The solids are placed in a vacuum oven at 45° C. and 28 inches of Hg vaccuum for 24 hours.

HPLC analysis on the filtrate indicates that the molar yield from the process is approximately 87%. Analysis of the solid samples withdrawn right after the completion of acid solution transfer and after the one-hour hold at 75° C., shows the hemi-fumarate salt. Final oven dried solids are also satisfactory. Approximate bulk density of the final solids is 0.28 g/cc.

### EXAMPLE 1(b)(i)

Fumaric acid (437 mg) is dissolved in 15 grams of IPA by heating to a jacket temperature of about 75° C. (reactor temperature about 72° C.) in a 100 ml jacketed reactor. In a separate reactor, the free base (19.97 grams as a 10% by weight/weight in IPA) solution is heated to the jacket temperature of about 75° C. Stirring is set at about 145 rpm in both the reactors. Once all the fumaric acid dissolves in the first reactor, this acid solution is transferred to the free-base solution through a transfer pipette, while maintaining the temperature at about 72° C. The transfer is complete within 10 minutes. Solids started precipitating out towards the end of the transfer. The slurry is held at about 75° C., for about 1 hour and cooled to about 20° C. over about twenty hours using a linear cooling ramp. The temperature is held at 20° C. for about one hour and then discharged on a 150 ml medium frit sintered glass funnel. The cake is washed with 10 ml of IPA and air dried for about two hours. The solids are placed in a vacuum oven at about 45° C. and 28 inches of Hg vaccuum for about 24 hours.

HPLC analysis on the filtrate indicates that the molar yield from the process is approximately 87%. Analysis of the solid samples withdrawn right after the completion of acid solution charge and after the one-hour hold at 75° C., showed them to be the hemi-fumarate salt. Final oven dried solids also satisfy all the attributes of the hemi-fumerate salt. Approximate bulk density of Example 1 (b)(i) is 0.256 g/cc.

### EXAMPLE 1(b)(ii)

Fumaric acid (437 mg) is dissolved in 15 grams of IPA by heating to a jacket temperature of about 75° C. (reactor temperature about 72° C.) in a 100 ml jacketed reactor. In a separate reactor 2.0 grams of crystalline free-base is dissolved in 20 grams of IPA by heating to the jacket temperature of about 75° C. Stirring is set at about 145 rpm in both the reactors. Once all the fumaric acid dissolves, the free-base solution is transferred to the acid solution through a transfer pipette, while maintaining the reactor temperature at about 72° C. The transfer is complete within 10 minutes. Solids start precipitating before the transfer is complete. The slurry is held at about 75° C., for about 1 hour and cooled to about 20° C. over about five hours using a linear cooling ramp. The reactor temperature is held at about 20° C. for about one hour and then discharged on a 150 ml medium frit sintered glass funnel. The cake is washed with 10 ml of IPA and air dried for about two hours. The solids are then placed in a vacuum oven at about 45° C. and 28 inches of Hg vaccuum for 24 hours.

HPLC analysis on the filtrate indicates that the molar yield from the process is approximately 95%. Analysis of the solid samples withdrawn right after the completion of acid solution transfer and after the one-hour hold at 75° C., shows them to be the hemi-fumarate salt. Final oven dried solids also satisfy all the attributes of the hemi-fumerate salt.

### EXAMPLE 1(b)(iii)

Fumaric acid (399 mg) is dissolved in 15 grams of IPA by heating to a jacket temperature of about 75° C. (reactor temperature about 72° C.) in a 100 ml jacketed reactor. In a separate reactor, 2.0 grams of crystalline free-base is dissolved in 20 grams of IPA by heating to the jacket temperature of about 75° C. Stirring is set at about 145 rpm in both the reactors. Once all the fumaric acid dissolves, the free-base solution is transferred to the acid solution through a transfer pipette over about 10 minutes, while maintaining the reactor temperature at about 72° C. Solids start precipitating out before the transfer is complete. The slurry is held at about 75° C., for about 1 hour and cooled to about 20° C. over about five hours using a linear cooling ramp. The reactor temperature is held at about 20° C. overnight and the reaction mixture is discharged on a 150 ml medium frit sintered glass funnel. The cake is washed with 10 ml of IPA and air dried for about two hours. The solids are then placed in a vacuum oven at about 45° C. and 28 inches of Hg vaccuum for 24 hours.

HPLC analysis on the filtrate indicates that the molar yield from the process is approximately 89%. Analysis of the solid samples withdrawn right after the transfer of the acid solution and after the one-hour hold at 75° C., showed them to be the hemi-fumarate salt. Final oven dried solids also satisfy all the attributes of the hemi-fumerate salt.

### EXAMPLE 1(b)(iv)

Fumaric acid (485 mg) is dissolved in 15 grams of IPA by heating to a jacket temperature of about 75° C. (reactor temperature about 72° C.) in a 100 ml jacketed reactor. In a separate reactor, 2.0 grams of crystalline free-base is dissolved in 20 grams of IPA by heating to the jacket temperature of about 75° C. Stirring is set at about 145 rpm in both the reactors. Once all the fumaric acid dissolves, the free-base solution is transferred to the acid solution through a transfer pipette over about 10 minutes, while maintaining the reactor temperature at about 72° C. Solids started precipitating out before the transfer is complete. The slurry is held at about 75° C., for about 1 hour and cooled to about 20° C. over about five hours using a linear cooling ramp. The reactor temperature is held at about 20° C. for about 1 hour and the reaction is discharged on a 150 ml medium frit sintered glass funnel. The cake is washed with 10 ml of IPA and air dried for about two hours. The solids are then placed in a vacuum oven at about 45° C. and 28 inches of Hg vaccuum for 24 hours.

HPLC analysis on the filtrate indicates that the molar yield from the process is approximately 91.5%. Analysis of the solid samples withdrawn right after transfer of the acid solution and after the one-hour hold at 75° C., showed them to be the hemi-fumarate salt. Final oven dried solids also satisfy all the attributes of the hemi-fumerate salt.

1. A fumarate salt of compound of the Formula I: 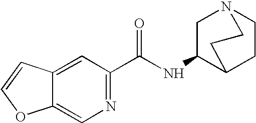
or pharmaceutical composition, racemic mixture, or pure enantiomer thereof, provided that the salt is the fumarate salt thereof.

2. The salt of claim 1, wherein the compound is a mono-fumarate salt of N-[(3R)-1-azabicyclo[2.2.2]oct-3-yl]furo[2,3-c]pyridine-5-carboxamide.

3. The salt of claim 2, wherein the salt is crystalline further having characteristic diffraction peaks at 18.90 and 24.97 degrees two-theta in a powder X-ray diffratction pattern.

4. The salt of claim 3, wherein the crystals have characteristic powder X-ray diffratction diffraction peaks at 18.21, 18.90, 21.74, and 24.97 degrees two-theta.

5. The salt of claim 2, wherein the salt has less than 0.3% water.

6. The salt of claim 5, wherein the salt has less than 0.2% water.

7. The salt of claim 6, wherein the salt has less than 0.1% water.

8. The salt of claim 1, wherein the compound is a hemi-fumarate salt of N-[(3R)-1-azabicyclo[2.2.2]oct-3-yl]furo[2,3-c]pyridine-5-carboxamide.

9. The salt of claim 8, wherein the salt is crystalline further having characteristic diffraction peaks at 19.84 and 24.83 degrees two-theta in a powder X-ray diffraction pattern.

10. The salt of claim 9, wherein the crystals have characteristic powder X-ray diffraction diffraction peaks 17.59, 18.43, 19.84, 22.74, and 24.83 degrees two-theta in a powder X-ray diffraction pattern.

11. The salt of claims 8, wherein the salt has less than 0.3% water.

12. The salt of claim 11, wherein the salt has less than 0.2% water.

13. The salt of claim 12, wherein the salt has less than 0.1% water.

14. A pharmaceutical composition comprising the fumarate salt of claim 1, and optionally an anti-psychotic agent.

15. A method for treating a disease or condition in a mammal in need thereof, wherein the mammal would receive symptomatic relief from the administration of a therapeutically effective amount of α7 nicotinic acetylcholine receptor agonist of claim 1.

16. The method of claim 15, wherein the salt is the mono-fumarate.

17. The method of claim 15, wherein the salt is the hemi-fumarate.

18. The method of claim 15, wherein the disease or condition is cognitive and attention deficit symptoms of Alzheimer's, neurodegeneration associated with diseases such as Alzheimer's disease, pre-senile dementia (mild cognitive impairment), senile dementia, schizophrenia, psychosis attention deficit disorder, attention deficit hyperactivity disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependant drug cessation, Gilles de la Tourette's Syndrome, glaucoma, neurodegeneration associated with glaucoma, or symptoms associated with pain.

19. The method of claim 18, wherein the salt is the mono-fumarate.

20. The method of claim 18, wherein the salt is the hemi-fumarate.

21. A preparation of mono-fumarate salt, comprising dissolving the free base in an alcohol by heating;
adding at least 1 eq of fumaric acid;
precipitating the salt out of solution; and
collecting, optionally washing the salt, and drying the salt.

22. The preparation of claim 21, further comprising that the alcohol is methanol or ethanol using a sufficient amount to give a concentration from about 0.04M to about 1M by heating.

23. The preparation o claim 21, further comprising that the alcohol is isopropanol using a sufficient amount to give a concentration of about 0.1M to about 1M.

24. The preparation of claim 23, further comprising the combining of the free base solution with a solution of at least 1 eq of fumaric acid dissolved in a second alcohol, optionally methanol or ethanol, to give a concentration of about 2M to about 5M, and further compsrising the addition of acetone to give a final concentration of about 0.1M to about 0.5M after the acid and free base solutions are combined.

25. The preparation of claim 24, further comprising a second addition of acetone to give a final concentration of about 0.05M to about 0.2M after about 1 to about 3 hours after the first addition of acetone, and further comprising the collection of the solid, optionally collection after about 8-24 hours of stirring, and optionally washing the resultant solid with acetone.

26. The preparation of claim 21, further comprising n-butanol as the alcohol using a sufficient amount to give a solution of about 0.6M to about 0.8M.

27. The preparation of claim 26, further comprising the addition of the free-base solution to a solution of about 0.35M to about 0.45M of at least 1 eq fumaric acid in 30% water/acetone.

28. The preparation of claim 27, further comprising concentrating the reaction to about 0.55M to about 0.75M, and further comprising the addition of sufficient amount of n-butanol to give a concentration of the free base from about 0.4M to about 0.6M.

29. The preparation of claim 21, wherein the salt has less than 0.3% water.

30. The preparation of claim 29, wherein the salt has less than 0.2% water.

31. The preparation of claim 30, wherein the salt has less than 0.1% water.

32. A preparation of hemi-fumarate salt, comprising dissolving the free base in an alcohol;
adding a solution of about 0.5 eq of fumaric acid dissolved in an alcohol;
adding the acid solution to the free-base solution;
collecting, optionally washing the salt, and drying the salt.

33. The preparation of claim 32, further comprising that the alcohol is isopropanol (IPA) at approximately 9 wt % by heating.

34. The preparation of claim 33, further comprising dissolving approximately 0.5 molar equivalents of fumaric acid in IPA by heating to approximately 70° C. to give a final concetration of about 2.8 wt %.

35. The preparation of claim 34, further comprising the combining of the free-base solution and the fumaric acid solution while maintaining the temperature.

36. The preparation of claim 35, further comprising allowing the system to cool to room temperature, filtering off the resulting solid, washing the solid with IPA and drying the solid.
